SEQUENCE LISTING (58 sequences)

SEQ ID NO: 1 - protein, 9 aa, Artificial sequence
  Homo sapiens source
EADPTGHSY

SEQ ID NO: 2 - protein, 9 aa, Artificial sequence
  Homo sapiens source
SAYGEPRKL

SEQ ID NO: 3 - protein, 9 aa, Artificial sequence
  Homo sapiens source
EVDPIGHLY

SEQ ID NO: 4 - protein, 9 aa, Artificial sequence
  Homo sapiens source
FLWGPRALV

SEQ ID NO: 5 - protein, 10 aa, Artificial sequence
  Homo sapiens source
MEVDPIGHLY

SEQ ID NO: 6 - protein, 12 aa, Artificial sequence
  Homo sapiens source
KISGGPRISYPL

SEQ ID NO: 7 - protein, 9 aa, Artificial sequence
  Homo sapiens source
ALSRKVAEL

SEQ ID NO: 8 - protein, 9 aa, Artificial sequence
  Homo sapiens source
AARAVFLAL

SEQ ID NO: 9 - protein, 8 aa, Artificial sequence
  Homo sapiens source
YRPRPRRY

SEQ ID NO: 10 - protein, 10 aa, Artificial sequence
  Homo sapiens source
SPSSNRIRNT

SEQ ID NO: 11 - protein, 9 aa, Artificial sequence
  Homo sapiens source
VLPDVFIRC

SEQ ID NO: 12 - protein, 9 aa, Artificial sequence
  Homo sapiens source
EEKLIVVLF

SEQ ID NO: 13 - protein, 9 aa, Artificial sequence
  Homo sapiens source
EEKLSVVLF

SEQ ID NO: 14 - protein, 10 aa, Artificial sequence
  Homo sapiens source
ACDPHSGHFV

SEQ ID NO: 15 - protein, 10 aa, Artificial sequence
  Homo sapiens source
ARDPHSGHFV

SEQ ID NO: 16 - protein, 9 aa, Artificial sequence
  Homo sapiens source
SYLDSGIHF

SEQ ID NO: 17 - protein, 9 aa, Artificial sequence
  Homo sapiens source
SYLDSGIHS

SEQ ID NO: 18 - protein, 9 aa, Artificial sequence
  Homo sapiens source
MLLAVLYCL

SEQ ID NO: 19 - protein, 9 aa, Artificial sequence
  Homo sapiens source
YMNGTMSQV

SEQ ID NO: 20 - protein, 9 aa, Artificial sequence
  Homo sapiens source
YMDGTMSQV

SEQ ID NO: 21 - protein, 9 aa, Artificial sequence
  Homo sapiens source
AFLPWHRLF

SEQ ID NO: 22 - protein, 9 aa, Artificial sequence
  Homo sapiens source
SEIWRDIDF

SEQ ID NO: 23 - protein, 9 aa, Artificial sequence
  Homo sapiens source
YEIWRDIDF

SEQ ID NO: 24 - protein, 15 aa, Artificial sequence
  Homo sapiens source
QNILLSNAPLGPQFP

SEQ ID NO: 25 - protein, 15 aa, Artificial sequence
  Homo sapiens source
DYSYLQDSDPDSFQD

SEQ ID NO: 26 - protein, 9 aa, Artificial sequence
  Homo sapiens source
ILTVILGVL

SEQ ID NO: 27 - protein, 9 aa, Artificial sequence
  Homo sapiens source
KTWGQYWQV

SEQ ID NO: 28 - protein, 9 aa, Artificial sequence
  Homo sapiens source
ITDQVPFSV

SEQ ID NO: 29 - protein, 9 aa, Artificial sequence
  Homo sapiens source
YLEPGPVTA

SEQ ID NO: 30 - protein, 10 aa, Artificial sequence
  Homo sapiens source
LLDGTATLRL

SEQ ID NO: 31 - protein, 10 aa, Artificial sequence
  Homo sapiens source
VLYRYGSFSV

SEQ ID NO: 32 - protein, 9 aa, Artificial sequence
  Homo sapiens source
LYVDSLFFL

SEQ ID NO: 33 - protein, 11 aa, Artificial sequence
  Homo sapiens source
SLLMWITQCFL

SEQ ID NO: 34 - protein, 9 aa, Artificial sequence
  Homo sapiens source
SLLMWITQC

SEQ ID NO: 35 - protein, 9 aa, Artificial sequence
  Homo sapiens source
QLSLLMWIT

SEQ ID NO: 36 - protein, 18 aa, Artificial sequence
  Homo sapiens source
HLYQGCQVVPLTSIISAV

SEQ ID NO: 37 - protein, 9 aa, Artificial sequence
  Homo sapiens source
LLGRNSFEV

SEQ ID NO: 38 - protein, 15 aa, Artificial sequence
  Rubella virus source
WVTPVIGSQARKCGL

SEQ ID NO: 39 - protein, 8 aa, Artificial sequence
  Rubella virus source
RVIDPAAQ

SEQ ID NO: 40 - protein, 15 aa, Artificial sequence
  Measles virus source
HQALVIKLMPNITLL

SEQ ID NO: 41 - protein, 9 aa, Artificial sequence
  Papilloma source
RLCVQSTHV

SEQ ID NO: 42 - protein, 9 aa, Artificial sequence
  Papilloma source
YVRDGNPYA

SEQ ID NO: 43 - protein, 10 aa, Artificial sequence
  Papilloma source
GYNKPLCDLL

SEQ ID NO: 44 - protein, 12 aa, Artificial sequence
  Influenza source
KGILGFVFTLTV

SEQ ID NO: 45 - protein, 13 aa, Artificial sequence
  Influenza source
EKYVKQNTLKLAT

SEQ ID NO: 46 - protein, 9 aa, Artificial sequence
  Hepatitis B source
WLSLLVPFV

SEQ ID NO: 47 - protein, 9 aa, Artificial sequence
  Hepatitis B source
FLGGTTVCL

SEQ ID NO: 48 - protein, 9 aa, Artificial sequence
  Hepatitis C source
YLVAYQATV

SEQ ID NO: 49 - protein, 9 aa, Artificial sequence
  Hepatitis C source
GLRDLAVAV

SEQ ID NO: 50 - protein, 14 aa, Artificial sequence
  Hepatitis C source
GYKVLVLNPSVAAT

SEQ ID NO: 51 - protein, 10 aa, Artificial sequence
  Hepatitis C source
KLVALGINAV

SEQ ID NO: 52 - protein, 15 aa, Artificial sequence
  Tetanus source
QYIKANSKFIGIYQL

SEQ ID NO: 53 - protein, 15 aa, Artificial sequence
  Homo sapiens source
TYELAPVFVLLEYVT

SEQ ID NO: 54 - protein, 15 aa, Artificial sequence
  Homo sapiens source
LKKMRFIIGWPGGSG

SEQ ID NO: 55 - protein, 15 aa, Artificial sequence
  Homo sapiens source
KKGAAAIGIGTDSVI

SEQ ID NO: 56 - protein, 15 aa, Artificial sequence
  Homo sapiens source
PLQCSALLVREEGLM

SEQ ID NO: 57 - protein, 15 aa, Artificial sequence
  Homo sapiens source
WLMWRAKGTTGFEAH

SEQ ID NO: 58 - protein, 16 aa, Artificial sequence
  Homo sapiens source
VIVMLTPLVEDGVKQC

(19) United States
(12) Patent Application Publication
(10) Pub. No.: US 2008/0063652 A1
(43) Pub. Date: Mar. 13, 2008

(54) Methods for Production of Regulatory T Cells and Uses Thereof

(75) Inventors: Mark J. Pykett, Boxford, MA (US); Michael Rosenzweig, Boston, MA (US)

Correspondence Address: WOLF GREENFIELD & SACKS, P.C., 600 ATLANTIC AVENUE, BOSTON, MA, 02210-2206, US

(73) Assignee: Cytomatrix, LLC, Chelmsford, MA (US)

(21) Appl. No.: 10/594,806

(22) PCT Filed: Mar. 29, 2005
(86) PCT No.: PCT/US05/10597

§ 371 Date: Sep. 18, 2007

Related U.S. Application Data
(60) Provisional application No. 60/557,669, filed on Mar. 29, 2004.

(51) Int. Cl. A61K 35/00; A61K 39/00; A61P 43/00; C12N 5/08

(52) U.S. Cl. 424/184.1; 424/93.71; 435/375

(57) ABSTRACT

The invention provides culture systems for the generation, expansion and isolation of regulatory T cells. These culture systems can be used to screen for factors that modulate regulatory T cell development. Regulatory T cells derived from such cultures can be used in various in vitro and in vivo applications, as can culture populations depleted of such cells.

### RELATED APPLICATIONS

This application claims priority under 35 U.S.C. § 119(e) to U.S. Provisional Application Ser. No. 60/557,669, filed Mar. 29, 2004, and entitled “METHODS FOR PRODUCTION OF REGULATORY T CELLS AND USES THEREOF,” the contents of which are herein incorporated by reference in their entirety.

### FIELD OF THE INVENTION

The invention pertains to the co-culture of hematopoietic progenitor cells and lymphoreticular stromal cells in three-dimensional devices, resulting in unexpectedly high numbers of regulatory T cells.

### BACKGROUND OF THE INVENTION

The generation of T cells occurs through a complex process in which stem cells from the bone marrow migrate to the thymus and undergo an ordered, sequential process differentiation into T cells. The developmental steps involved in this process can be distinguished by the expression of certain molecules on the surface of T cells, including the T cell receptor and the co-receptors CD4 and CD8. These cells pass through a number of check points during the development process. In general terms, the cells that can distinguish diseased cells from healthy cells survive and emigrate to the peripheral circulation where they can encounter viral or bacterial proteins and be called into action to either kill the infected cells or recruit help from other cells to fight the infection. However, not all potentially auto-reactive T cells are deleted during T cell development as they can be readily isolated from lymphoid tissue.

The thymus has been shown to be an obligatory factor in T cell differentiation of hematopoietic cells and appears necessary for traditional T cell generation as well as the generation of regulatory T cells. It has been documented that the presence of a three dimensional organ is required, as in vitro models that do not include the thymus and a three dimensional structure fail to support T cell lymphopoiesis (Owen J J, et al., Br Med Bull., 1989, 45:350-360).

Primitive hematopoietic progenitors in the fetal liver or bone marrow give rise to lineage committed cells, including progenitors committed to the T lymphoid lineage. These most immature cells are identified by the surface expression of CD34. Cells committed to the T cell lineage also express CD34, but no discrete expression of other epitopes found only on T cell progenitors has been described. Further, T lymphocyte differentiation normally occurs via a series of discrete developmental stages. Primitive progenitor cells which do not express lymphocyte specific cell surface markers (CD34+ CD3− CD4− CD8−) migrate to the thymus where they acquire, through a series of maturational events, the phenotype CD34− CD3− CD4+ CD8−. These cells then mature into double positive CD4+ CD8+ cells, most of which are CD3+, although CD3 expression is not universally detectable. These cells further undergo both positive and negative selection, and mature to develop into single positive T cells (CD4+ CD8− or CD4− CD8+). These cells ultimately migrate into the peripheral circulation as naive T cells.

The description of a subpopulation of T cells that suppress immune responses has been hypothesized since the 1970s. Recent advances have identified such a population of T cells, characterized as regulatory T cells. This concept extends the understanding of the maintenance of peripheral tolerance via suppression of effector functions as an independent process from anergy and T cell deletion. These thymus-derived, regulatory T cells have been characterized phenotypically as CD4⁺CD25⁺(Martin et al, J I 2004; Baecher-Allan et al J I 2001; Dubois et al Blood 2003). In addition, the majority of these cells express CD45RO, although they are not memory T cells (Martin et al 2004).

The study and therapeutic use of regulatory T cells is frustrated by an inability to generate reasonable numbers of such cells in vitro. Accordingly, there exists a need for methods and culture systems for generating regulatory T cells.

### SUMMARY OF THE INVENTION

The invention relates, in part, to in vitro culture of regulatory T cells. The invention provides methods and culture systems for generation, growth and expansion of regulatory T cells from various hematopoietic progenitor populations.

The culture system utilizes biocompatible, open-pore, three-dimensional matrices, and uses human and non-human lymphoreticular stromal cells (including fibroblasts, keratinocytes, macrophages, dendritic cells, epithelial cells, all of which may be derived from neuroectodermal tissues including skin, thymus and gastro-intestinal tissues), to provide the appropriate conditions for the expansion and differentiation of human and non-human hematopoietic progenitor cells toward a specific T cell population that is CD4+CD25+.

This system provides significant advantages over existing techniques. For example, it can provide for the rapid generation of a large number of differentiated progeny necessary for laboratory analysis and/or therapeutic uses, including in vivo reinfusion of regulatory T cells (possibly in the absence of other culture progeny) or re-infusion of culture progeny depleted of regulatory T cells.

Surprisingly, according to the invention, it has been discovered that hematopoietic progenitor cells co-cultured with lymphoreticular stromal cells in a porous solid scaffold, generate at a fast rate an unexpectedly high number of differentiated progeny of a lymphoid-specific lineage including a percentage of regulatory T cells. Also surprising, according to the invention, is the discovery that lesser amounts of nonlymphoid cells (i.e. myelo-monocytic cells) are generated from the co-culture of hematopoietic progenitor cells and lymphoreticular stromal cells in a porous solid scaffold of the invention when compared to existing methods. Thus, the present invention permits for the rapid generation of a large number of differentiated, regulatory T cells from a relatively small number of hematopoietic progenitor cells. Such results were never before realized using known art methodologies (e.g., as in U.S. Pat. No. 5,677,139 by Johnson et al., which describes the in vitro differentiation of CD3+ cells on primate thymic stroma monolayers, or as in U.S. Pat. No. 5,541,107 by Naughton et al., which describes a three-dimensional bone marrow cell and tissue culture system).

Thus, in one aspect, the invention provides a method for in vitro production of regulatory T cells, comprising introducing an amount of hematopoietic progenitor cells and an amount of lymphoreticular stromal cells capable of mitosis into an open cell porous, solid matrix having interconnected pores of a pore size sufficient to permit the hematopoietic progenitor cells and the lymphoreticular stromal cells to grow in the matrix, co-culturing the hematopoietic progenitor cells and the lymphoreticular stromal cells, and isolating regulatory T cells from the cultured cells.

In another aspect, the invention provides a method for producing a hematopoietic cell population depleted of regulatory T cells, comprising introducing an amount of hematopoietic progenitor cells and an amount of lymphoreticular stromal cells capable of mitosis into an open cell porous, solid matrix having interconnected pores of a pore size sufficient to permit the hematopoietic progenitor cells and the lymphoreticular stromal cells to grow in the matrix, co-culturing the hematopoietic progenitor cells and the lymphoreticular stromal cells, and removing regulatory T cells from the cultured cells to produce a hematopoietic cell population depleted of regulatory T cells.

Various embodiments apply equally to these and other aspects of the invention and these are recited below. In one embodiment, the lymphoreticular stromal cells are derived from at least one lymphoid soft tissue selected from the group consisting of thymus, spleen, liver, lymph node, skin, tonsil, Peyer's patches and combinations thereof, and comprises one or more of fibroblasts, keratinocytes, epithelial cells, dendritic cells (DCs), and antigen presenting cells; and the amount of the lymphoreticular stromal cells is sufficient to support the growth and differentiation of the hematopoietic progenitor cells.

In another embodiment, the hematopoietic progenitor cells and the lymphoreticular stromal cells are co-cultured in the presence of IL-7 and IL-15.

The hematopoietic progenitor cells and the lymphoreticular stromal cells may both be of human origin or they may both be of murine origin, but they are not so limited.

In one embodiment, the regulatory T cells are isolated or removed (depending on the aspect of the invention) based on a CD4+CD25+ phenotype. Isolation or removal of regulatory T cells may be effected by using fluorescent activated cell sorting, affinity column separation, affinity magnetic beads, affinity magnetic particles, complement-mediated lysis, panning, or tetrameric complex based separation.

In one embodiment, the hematopoietic progenitor cells are selected from the group consisting of pluripotent stem cells, multipotent progenitor cells and progenitor cells committed to specific hematopoietic lineages. In another embodiment, the hematopoietic progenitor cells are derived from tissue selected from the group consisting of bone marrow, peripheral blood, mobilized peripheral blood, umbilical cord blood, placental blood, lymphoid soft tissue, fetal liver, embryonic cells and aortal-gonadal-mesonephros derived cells. In an important embodiment, the hematopoietic progenitor cells are derived from tissue selected from the group consisting of bone marrow, mobilized peripheral blood and umbilical cord blood.

In one embodiment, the lymphoreticular stromal cells are seeded prior to inoculating the hematopoietic progenitor cells.

In one embodiment, the porous solid matrix is an open cell porous matrix having a percent open space of at least 75%. In another embodiment, the porous solid matrix is an open cell porous matrix having at least 80 pores per square inch (ppi). In related embodiments, the porous solid matrix has pores defined by interconnecting ligaments having a diameter at midpoint, on average, of less than 150 μm. The porous, solid matrix having seeded hematopoietic progenitor cells and their progeny, and lymphoreticular stromal cells, may be impregnated with a gelatinous agent that occupies pores of the matrix. The preferred embodiments of the invention are solid, unitary macrostructures, i.e. not beads or packed beads. They also comprise nonbiodegradable materials.

In one embodiment, the porous solid matrix is a metal-coated reticulated open cell foam of carbon containing material. In another embodiment, the metal is selected from the group consisting of tantalum, titanium, platinum, niobium, hafnium, tungsten, and combinations thereof, and wherein said metal is coated with a biological agent selected from the group consisting of collagens, fibronectins, laminins, integrins, glycosaminoglycans, vitrogen, antibodies and fragments thereof, and combinations thereof. Preferably, the metal is tantalum.

In one embodiment, the lymphoid soft tissue is selected from the group consisting of thymus and skin. Preferably the skin stroma comprises fibroblasts and keratinocytes. In a related embodiment, the progenitor cells committed to specific hematopoietic lineages are committed to a T cell lineage. In important embodiments, the hematopoietic progenitor cells are CD34+ cells. In certain embodiments, the progenitor cells are CD34+ cells, the lymphoreticular stromal cells are derived from skin, and the co-culture comprises IL-7 and IL-15. In related embodiments, the hematopoietic progenitor cells and the lymphoreticular stromal cells are autologous to a subject to be treated with the isolated regulatory T cells. In other related embodiments, the hematopoietic progenitor cells are allogeneic and the lymphoreticular stromal cells are autologous to a subject to be treated with the isolated regulatory T cells.

In some embodiments, the method further comprises exposing the culture to antigen or antigen presenting cells.

In yet other embodiments, the method further comprises isolating antigen-specific T cells from the hematopoietic cell population depleted of regulatory T cells.

In yet another aspect, the invention provides a method for inhibiting an immune response, comprising administering to a subject in need thereof isolated regulatory T cells produced according to any of the afore-mentioned methods, in an amount effective to inhibit an immune response.

In one embodiment, the isolated regulatory T cells are administered systemically. In another embodiment, the isolated regulatory T cells are administered locally to a site of inflammation.

In one embodiment, the subject has undergone or is undergoing a transplantation. The transplantation may be a bone marrow or an organ or a cell transplantation.

In another embodiment, the subject has an inflammatory condition. The inflammatory condition may be selected from the group consisting of non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis, and an abscess.

In another embodiment, the inflammatory condition is an autoimmune condition. The autoimmune condition may be selected from the group consisting of rheumatoid arthritis, rheumatic fever, ulcerative colitis, Crohn's disease, autoimmune inflammatory bowel disease, insulin-dependent diabetes mellitus, diabetes mellitus, juvenile diabetes, spontaneous autoimmune diabetes, gastritis, autoimmune atrophic gastritis, autoimmune hepatitis, thyroiditis, Hashimoto's thyroiditis, insulitis, oophoritis, orchitis, uveitis, phacogenic uveitis, multiple sclerosis, myasthenia gravis, primary myxoedema, thyrotoxicosis, pernicious anemia, autoimmune haemolytic anemia, Addison's disease, scleroderma, Goodpasture's syndrome, Guillain-Barre syndrome, Graves' disease, glomerulonephritis, psoriasis, pemphigus vulgaris, pemphigoid, sympathetic opthalmia, idiopathic thrombocylopenic purpura, idiopathic feucopenia, Siogren's syndrome, Wegener's granulomatosis, poly/dermatomyositis, and systemic lupus erythematosus.

In another embodiment, the subject has a microbial infection, such as but not limited to an RSV infection. In another embodiment, the microbial infection results in sepsis.

In one embodiment, the subject has an allergy or is experiencing an allergic reaction. The subject may be experiencing immune hypersensitivity, for example.

In another embodiment, the subject has stromal keratitis. In another embodiment, the subject has atherosclerosis. In another embodiment, the subject has myocarditis

In one embodiment, the subject is undergoing gene therapy.

In another embodiment, the subject is undergoing allograft rejection.

In one embodiment, the progenitor cells and lymphoreticular stromal cells are autologous to the subject. In another embodiment, the progenitor cells are allogeneic and the lymphoreticular stromal cells are autologous to the subject.

In one embodiment, the isolated regulatory T cells are administered to the subject repeatedly.

In another aspect, the invention provides a method for increasing immune reactivity of a transplanted cell population, comprising administering to a subject in need thereof a cell population depleted of regulatory T cells.

In one embodiment, the cell population depleted of regulatory T cells is the hematopoietic cell population depleted of regulatory T cells produced according to the methods described above.

In one embodiment, the transplanted cell population is a hematopoietic cell population.

In another embodiment, the subject is undergoing cancer treatment such as leukemia treatment.

In another embodiment, the transplanted cell population is a dendritic cell based vaccine or an antigen presenting cell based vaccine.

In yet another embodiment, the transplanted cell population is an antigen-specific effector T cell population.

In yet another aspect, the invention provides an isolated or enriched population of regulatory T cells produced by the afore-mentioned methods.

In a further aspect of the invention, a method for identifying regulatory T cell modulating agents is provided. The method involves introducing an amount of hematopoietic progenitor cells and an amount of lymphoreticular stromal cells into a porous, solid matrix having interconnected pores of a pore size sufficient to permit the hematopoietic progenitor cells and the lymphoreticular stromal cells to grow throughout the matrix, co-culturing the hematopoietic progenitor cells and the lymphoreticular stromal cells in the presence of at least one candidate regulatory T cell modulating agent (in a test co-culture), and determining whether the at least one candidate agent affects regulatory T cell development generation in the test co-culture by comparing the test co-culture regulatory T cell generation to a control co-culture wherein hematopoietic progenitor cells and lymphoreticular stromal cells are co-cultured in the absence of the at least one candidate agent. Various embodiments are provided, wherein the hematopoietic progenitor cells, the lymphoreticular stromal cells, and the porous solid matrix have one or more of the preferred characteristics as described above, and the cells are cultured as described above. In preferred embodiments the lymphoreticular stromal cells are thymic stromal cells.

These and other aspects of the invention, as well as various advantages and utilities, will be more apparent with reference to the detailed description of the preferred embodiments.

### BRIEF DESCRIPTION OF THE FIGURES

FIG. 1 shows the immunophenotype of T cells recovered from Cytomatrix® cultures and demonstrates the existence of CD4+CD25+ T cells which are indicative of regulatory T cells. Cells were isolated from the Cytomatrix® cultures and stained with fluorescent labeled antibodies followed by flow cytometry analyses. CD3+CD4+ expression is shown.

FIG. 2A shows the CD3/CD4 immunophenotype of T cells recovered from Cytomatrix® cultures. Cells were isolated from the Cytomatrix® cultures and stained with fluorescent labeled antibodies followed by flow cytometry analyses.

FIG. 2B demonstrates CD25 and CD45RO expression on CD4+CD3+ T cells indicative of regulatory T cells. Using gating, CD25 and CD45RO co-expression is demonstrated on the CD3+CD4+ cells. The sized of the CD25+ CD45RO population varies from 30% to 70%.

It is to be understood that the Figures are not required for enablement of the invention.

### DETAILED DESCRIPTION OF THE INVENTION

Effector T cells such as self-reactive T cells can be regulated by a subset of thymus derived T cells known as regulatory T cells. This mechanism appears to be critical in the maintenance of peripheral tolerance and the loss of this subset of T cells may be associated with an increase in a number of disease states linked to immune dysregulation.

The invention involves the unexpected discovery that hematopoietic progenitor cells co-cultured with lymphoreticular stromal cells in a porous solid scaffold, generate at a fast rate an unexpectedly high number of regulatory T cells. The present invention permits in part the rapid generation and isolation of a large number of regulatory T cells from a relatively small number of hematopoietic progenitor cells.

The invention further provides methods related to administration of regulatory T cells into subjects in need of immune inhibition. For example, the regulatory T cells can be infused into subjects in order to induce tolerance. Such subjects include but are not limited to those experiencing abnormal (i.e., as used herein, above normal) immune responses, such as autoimmune responses. The invention also provides methods related to administration of cell populations that are depleted of regulatory T cells. Accordingly, the invention contemplates the use of these various cell populations inter alia in transplantation, implantation, and/or infectious diseases.

“Regulatory T cells” as used herein are CD4+CD25+ cells that exhibit immunoinhibitory properties. These cells can thus be characterized phenotypically or functionally. Prior to the invention, they were primarily obtained from peripheral blood, where they constitute approximately 10% of T cells. The cells generated in vitro according to the invention are substantially similar (e.g., in properties and function) to the cells produced naturally in vivo. Regulatory T cells can also be identified based on CD4+CD45RO+ cell surface expression.

The invention in one aspect involves culturing hematopoietic cells in a porous solid matrix, in the absence of exogenous growth agents, to produce lymphoid tissue origin regulatory T cells. A porous, solid matrix, is defined as a three-dimensional structure with “sponge-like” continuous pores forming an interconnecting network. The matrix can be rigid or elastic, and it provides a scaffold upon which cells can grow throughout. Its pores are interconnected and provide the continuous network of channels extending through the matrix and also permit the flow of nutrients throughout. A preferred matrix is an open cell foam matrix having a percent open space of at least 50% and preferably 75%. In one important embodiment, the matrix has a percent open space of approximately 85%. The matrix can also be defined by the number of pores per square inch (ppi). In some embodiments, the matrix is one having 80 ppi. It is preferred that the open space comprise the majority of the matrix. This is believed to maximize cell migration, cell-cell contact, space for cell growth and accessibility to nutrients. It is preferred that the porous matrix be formed of a reticulated matrix of ligaments which at their center point are less than 150 μm in diameter, preferably 60 μm, whereby a cell can reside on or interact with a portion of the ligament. Preferably, the average pore diameter is on the order of 300 μm, which resembles cancellous bone.

Suitable matrices can be obtained using a number of different methods. Examples of such methods include solvent casting or extraction of polymers, track etching of a variety of materials, foaming of a polymer, the replamineform process for hydroxyapatite, and other methodologies well known to those of ordinary skill in the art. The materials employed can be natural or synthetic, including biological materials such as proteins, hyaluronic acids, synthetic polymers such as polyvinyl pyrolidones, polymethylmethacrylate, methyl cellulose, polystyrene, polypropylene, polyurethane, ceramics such as tricalcium phosphate, calcium aluminate, calcium hydroxyapatite and ceramic-reinforced or coated polymers. If the starting material for the scaffold is not metal, a metal coating can be applied to the three-dimensional matrix. Metal coatings provide further structural support and/or cell growth and adhesive properties to the matrix. Preferred metals used as coatings comprise tantalum, titanium, platinum and metals in the same element group as platinum, niobium, hafnium, tungsten, and combinations of alloys thereof. Coating methods for metals include a process such as CVD (Chemical Vapor Deposition).

The preferred matrix, referred to herein as Cytomatrix® (Cytomatrix, Woburn, Mass.), is described in detail in U.S. Pat. No. 5,282,861 (incorporated herein by reference). More specifically, the preferred matrix is a reticulated open cell substrate formed by a lightweight, substantially rigid foam of carbon-containing material having open spaces defined by an interconnecting network, wherein said foam material has interconnected continuous channels, and a thin film of metallic material deposited onto the reticulated open cell substrate and covering substantially all of the interconnecting network to form a composite porous biocompatible material creating a porous microstructure similar to that of natural cancellous bone.

Additionally, such matrices can be coated with biological agents which can promote cell adhesion for the cultured hematopoietic progenitor cells, allowing for improved migration, growth and proliferation. Preferred biological agents comprise collagens, fibronectins, laminins, integrins, glycosaminoglycans, vitrogen, antibodies and fragments thereof, functional equivalents of these agents, and combinations thereof.

“Hematopoietic progenitor cells” as used herein refers to immature blood cells having the capacity to self-renew and to differentiate into the more mature blood cells (also described herein as “progeny”) comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). It is known in the art that such cells may or may not include CD34+ cells. CD34+ cells are immature cells present in blood products, that express the CD34 cell surface marker, and are believed to include a subpopulation of cells with “progenitor cell” properties as defined above. It is well known in the art that hematopoietic progenitor cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, including those of regulatory T cell lineage, B cell lineage, dendritic cell lineage, Langerhans cell lineage and/or lymphoid tissue-specific macrophage cell lineage. Progenitors committed to specific hematopoietic lineages include CD34+CD7+ cells that are committed to the T cell lineage. This latter cell type or CD34+ cells can be used to initiate the cell cultures of the invention.

The hematopoietic progenitor cells can be obtained from blood products. A “blood product” as used in the present invention defines a product obtained from the body or an organ of the body containing cells of hematopoietic origin. Such sources include unfractionated bone marrow, umbilical cord, peripheral blood (including mobilized peripheral blood), liver, thymus, lymph and spleen. It will be apparent to those of ordinary skill in the art that all of the aforementioned crude or unfractionated blood products can be enriched for cells having “hematopoietic progenitor cell” characteristics in a number of ways. For example, the blood product can be depleted of the more differentiated progeny. The more mature, differentiated cells can be selected against, via cell surface molecules they express. Additionally, the blood product can be fractionated by selecting for CD34+ cells. As mentioned earlier, CD34+ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

In important embodiments, the progenitor cells are derived, particularly in the human system, from bone marrow, peripheral blood (e.g., following mobilization therapy, as is known in the art), or cord blood.

The cells co-cultured with the hematopoietic progenitor cells according to the methods of the invention are lymphoreticular stromal cells. “Lymphoreticular stromal cells” as used herein may include, but are not limited to, all cell types present in a lymphoid tissue which are not lymphocytes or lymphocyte precursors or progenitors, e.g., epithelial cells, endothelial cells, mesothelial cells, dendritic cells, splenocytes and macrophages. Lymphoreticular stromal cells also include cells that would not ordinarily function as lymphoreticular stromal cells, such as fibroblasts and/or keratinocytes (both of which are present in the skin stroma population), which may or may not have been genetically altered to secrete or express on their cell surface the factors necessary for the maintenance, growth and/or differentiation of hematopoietic progenitor cells, including their progeny.

Lymphoreticular stromal cells are derived from the disaggregation of a piece of lymphoid tissue (see discussion below and the Examples). Such cells according to the invention are capable of supporting in vitro the maintenance, growth and/or differentiation of hematopoietic progenitor cells, including their progeny. Together with IL-7 and IL-15, these stromal cells are now also involved in the generation of regulatory T cells, particularly from progenitor cells such as CD34+ cells. By “lymphoid tissue” it is meant to include bone marrow, peripheral blood (including mobilized peripheral blood), umbilical cord blood, placental blood, fetal liver, embryonic cells (including embryonic stem cells), aortal-gonadal-mesonephros derived cells, and lymphoid soft tissue. “Lymphoid soft tissue” as used herein includes, but is not limited to, tissues such as thymus, spleen, liver, lymph node, skin, tonsil, adenoids and Peyer's patch, and combinations thereof.

In some embodiments, the lymphoreticular stromal cells are skin or thymic stromal cells of human origin and the hematopoietic progenitor cells are of human origin. Preferably, the stromal cells are autologous to the subject being treated (i.e., derived from the subject being treated). The source of the progenitor cells however will depend on the condition being treated with the regulatory T cells (or the cultured cells depleted of such regulatory T cells). For example, if the condition is an autoimmune condition, then preferably the progenitor cells are also autologous to the subject being treated. If instead the condition is graft versus host disease (or the likelihood thereof), then the progenitor cells are non-autologous but would preferably still be allogeneic (i.e., derived from another subject of the same species). In human transplant settings, preferably, allogeneic cells are typed in order to match major and minor MHC loci, as is routinely practiced in the art.

Generally, cultures are established with allogeneic cells (i.e., the progenitor and stromal cells derive from the same species, if not necessarily the same subject). For example, if murine regulatory T cells were desired, the culture would preferably be inoculated with murine stromal cells and murine progenitor cells and most commonly these would be syngeneic. In a clinical setting, cultures would preferably be inoculated with human stromal cell and human progenitor cells.

Lymphoreticular stromal cells provide the supporting microenvironment in the intact lymphoid tissue for the maintenance, growth and/or differentiation of hematopoietic progenitor cells, including their progeny. The microenvironment includes soluble and cell surface factors expressed by the various cell types which comprise the lymphoreticular stroma. Generally, the support which the lymphoreticular stromal cells provide may be characterized as both contact-dependent and non-contact-dependent.

As mentioned herein, lymphoreticular stromal cells may be autologous (“self ”) or non-autologous (“non-self,” e.g., allogeneic, syngeneic or xenogeneic) with respect to hematopoietic progenitor cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Lymphoreticular stroma cells may be obtained from the lymphoid tissue of a human or a non-human subject at any time after the organ/tissue has developed to a stage (i.e., the maturation stage) at which it can support the maintenance growth and/or differentiation of hematopoietic progenitor cells. The stage will vary between organs/tissues and between subjects. In primates, for example, the maturation stage of thymic development is achieved during the second trimester. At this stage of development the thymus can produce peptide hormones such as thymulin, alpha, and beta₄-thymosin, and thymopoietin, as well as other factors required to provide the proper microenvironment for T cell differentiation. The different maturation stages for the different organs/tissues and between different subjects are well known in the art.

The lymphoid tissue from which lymphoreticular stromal cells are derived usually determines the lineage-commitment hematopoietic progenitor cells undertake, resulting in the lineage-specificity of the differentiated progeny. In certain embodiments, the lymphoreticular stromal cells are skin or thymic stromal cells and the multipotent progenitor cells and/or committed progenitor cells are CD34+ cells and/or progenitor cells committed to the T cell lineage. In other embodiments, the lymphoreticular stromal cells are skin stromal cells and include, among other cells, fibroblasts, keratinocytes, epithelial cells, DCs and/or macrophages.

Various other embodiments are provided, wherein the lymphoreticular stromal cells may be genetically altered. The lymphoreticular stromal cells may be transfected with exogenous DNA that encodes, for example, a hematopoietic growth factor such as but not limited to IL-7 or IL-15, or any other regulatory T cell factor.

As mentioned earlier, lymphoreticular stromal cells are derived from the disaggregation of a piece of lymphoid tissue or skin, forming cell suspensions. These lymphoreticular stromal cell suspensions may be used directly, or made non-mitotic by procedures standard in the tissue culture art. Examples of such methods are irradiation of lymphoreticular stromal cells with a gamma-ray source or incubation of the cells with mitomycin C for a sufficient amount of time to render the cells mitotically inactive. In some embodiments, it is preferable not to treat the stromal cells with either irradiation or mitomycin C. These cells are referred to as “capable of mitosis” although they may not necessarily undergo mitosis during all or most of the co-culture period due to contact inhibition.

The lymphoreticular stromal cells are seeded into the three-dimensional matrix of the invention and permitted to attach to a surface of the porous, solid matrix. In preferred embodiments, the stromal cells are introduced prior to the progenitor cells and allowed to grow to confluence (or near confluence). Once confluent, the stromal cells generally cease to divide due to contact inhibition. At or about this time, the progenitor cells are introduced into the cultures. Establishment of stromal confluence can take about 2 weeks on average, and generation of regulatory T cells can take another 2 weeks on average following introduction of the progenitor cells, in some embodiments.

It should be noted that the lymphoreticular stromal cells may alternatively be cryopreserved for later use or for storage and shipment to remote locations, such as for use in connection with the sale of kits. Cryopreservation of cells cultured in vitro is well established in the art. Subsequent to isolation of a cell sample, cells may be cryopreserved by first suspending the cells in a cryopreservation medium and then gradually freezing the cell suspension. Frozen cells are typically stored in liquid nitrogen or at an equivalent temperature in a medium containing serum and a cryopreservative such as dimethyl sulfoxide.

According to some aspects of the invention, hematopoietic progenitor cells and lymphoreticular stromal cells are co-cultured in one of the foregoing porous solid matrices, in the absence of exogenous growth agents, to produce regulatory T cells. In some important embodiments, however, the stromal cell and progenitor cells are co-cultured in the presence of at least IL-15, and more preferably IL-7 and IL-15. When used, these interleukins are introduced into the culture preferably together and at the substantially the same time as the progenitor cells (i.e., within an hour of each other). In other embodiments, however, they may be administered after the introduction of progenitor cells and at different times from each other and in different sequence. Progenitor cells are generally cultured in the presence of the stromal cells for an average of 14 days, although this is not intended to be limiting.

The co-culture of the hematopoietic progenitor cells (and progeny thereof) with lymphoreticular stromal cells preferably occurs under conditions sufficient to produce a T cells and more preferably regulatory T cells from the hematopoietic progenitor cells. Some of the conditions employed are known in the art (e.g., temperature, CO₂ and O₂ content, nutritive media, etc.). The time sufficient to achieve the desired number of regulatory T cells (and in some instances the desired number of antigen-specific effector T cells) is a time that can be determined by a person skilled in the art, particularly with reference to the invention and the Examples provided. The time may generally vary depending on, inter alia, the original number of cells seeded. The amounts of hematopoietic progenitor cells and lymphoreticular stromal cells initially introduced (and subsequently seeded) into the porous solid matrix may vary according to the needs of the experiment. The ideal amounts can be easily determined by a person skilled in the art in accordance with needs and with the teaching provided herein. As mentioned above, preferably, the lymphoreticular stromal cells would form a confluent or semi-confluent layer onto the matrix. Confluence can be indicated by discoloration of the media over a certain period of time. Hematopoietic progenitor cells may be added at different times and in different numbers, depending on the particular application.

In certain embodiments of the invention the porous solid matrix having seeded hematopoietic progenitor cells, with or without their progeny, and lymphoreticular stromal cells is impregnated with a gelatinous agent that occupies pores of the matrix. The hematopoietic progenitor cells, with or without their progeny, and/or the lymphoreticular stromal cells can be seeded prior to, substantially at the same time as, or following impregnation (or infiltration) with a gelatinous agent. For example, the cells may be mixed with the agent and seeded at the same time as the impregnation of the matrix with the agent. In some embodiments, the cells are seeded onto the porous solid matrix prior to application of the agent. In certain embodiments the lymphoreticular stromal cells are seeded in a similar manner. A person of ordinary skill in the art can easily determine seeding conditions. Preferably the lymphoreticular stromal cells are seeded prior to the hematopoietic progenitor cells and prior to impregnation with the agent.

“Impregnation” with a gelatinous agent can serve, inter alia, to contain the cells within the matrix, or to help maintain and/or enhance cell attachment onto the matrix. The “gelatinous” agent may be one that can be maintained in a fluid state initially (i.e., “gelable”), and after its application into the matrix, be gelatinized in situ in the matrix. Such gelatinization may occur in a number of different ways, including altering the agent's temperature, irradiating the agent with an energy source (e.g., light), etc. The “gelatinous” agent also is characterized by its ability to allow the nutrients of the growth media to reach the cells throughout the matrix. Exemplary “gelatinous” agents include cellulosic polysaccharides (such as cellulose, hemicellulose, methylcellulose, and the like), agar, agarose, albumin, algal mucin, mucin, mucilage, collagens, glycosaminoglycans, and proteoglycans (including their sulphated forms). In certain embodiments, the gelatinous agent may impregnate the matrix completely, in some embodiments partially, and in other embodiments minimally, serving only as a coating of all or some of the outer surfaces of the matrix. In important embodiments where gelatinous agents are employed, the “gelatinous” agent is methylcellulose and the impregnation is complete.

The cells present after or during the co-culture of progenitor and stromal cells are referred to as “cultured cells”. The cultured cells are comprised of regulatory T cells, effector T cells (including antigen-specific effector T cells) and cells of various other lymphoid and myeloid lineages, as well as progenitor and stromal cells that initiated the culture. It has been discovered according to the invention that co-culture of progenitors and stromal cells, particularly in the presence of IL-7 and IL-15 leads to the generation of a greater proportion of CD4+ cells and a greater proportion of regulatory T cells, than is observed in peripheral blood. This increase appears to be associated with a decrease in the number and frequency of myeloid committed cultured cells.

According to various aspects of the invention, the cultured cells are harvested and regulatory T cells are isolated away from the other cultured cells.

“Harvesting” the cultured cells is defined as the dislodging or separation of cells from the matrix. This can be accomplished using a number of methods, such as enzymatic and non-enzymatic, centrifugal, electrical or by size, or by flushing of the cells using the media in which the cells are incubated. In some embodiments, only non-adherent cells are harvested from the cultures and this can be achieved via flushing of media or wash solutions through the matrices, optionally with slight agitation. The harvested cells can be further separated either to isolate or deplete regulatory T cells, as well as other cell types, if additionally desired.

Alternatively, regulatory T cells may be removed from the cultured cells resulting in a “hematopoietic cell population depleted of regulatory T cells”. This latter population generally contains all remaining cells from the co-culture, although it may be further processed to enrich or deplete other cell types as well. This population can be used inter alia in transplantation or vaccination settings, as described in greater detail herein. Removal of regulatory T cells embraces physical separation of these cells from the remaining cells as well as eradication of these cells while in the population (e.g., via complement-mediated lysis). The resultant populations can then be further manipulated depending on the application. Further processing may involve selection of antigen-specific effector T cells, as an example.

Regulatory T cells can be isolated in a number of ways. In one embodiment, regulatory T cells are isolated phenotypically according to their cell surface phenotype (i.e., CD4+CD25+). Such isolation can be effected in a number of ways including but not limited to fluorescent activated cell sorting (FACS), affinity column separations, affinity bead or magnetic particle separations, and the like. These methods can similarly be used to deplete the cultured cell populations of regulatory T cells, as described in greater detail herein.

Regulatory T cells can also be isolated by negatively selecting against cells that are not regulatory T cells. This can be accomplished for example using the afore-mentioned procedures. As an example, these cells can be removed by performing a lineage depletion. Lineage depletion can be accomplished by labeling cells with antibodies for particular lineages such as the B lineage (e.g., antibodies to CD19 or CD20, in the human system), the macrophage/monocyte lineage (e.g., antibodies to CD14, in the human system), the cytotoxic T cell lineage (e.g., antibodies to CD8), the granulocyte lineages, the erythrocytes lineages, the megakaryocytes lineages, and the like. Markers for these specific lineages are listed herein. Cells labeled with one or more lineage specific antibodies can then be removed either by affinity column processing (where the lineage marker positive cells are retained on the column), by affinity magnetic beads or particles (where the lineage marker positive cells are attracted to the separating magnet), by panning (where the lineage marker positive cells remain attached to the secondary antibody coated surface), or by complement mediated lysis (where the lineage marker positive cells are lysed in the presence of complement by virtue of the antibodies bound to their cell surface). These and other depletion methodologies are known in the art.

Another lineage depletion strategy involves tetrameric complex formation. Cells are isolated using tetrameric anti-human antibody complexes (e.g., complexes specific for CD8, CD14, CD16, CD19, CD24, CD56, CD66b, CD41, CD33, CD11b, CD15, and Glycophorin-A) and magnetic colloid in conjunction with StemSep columns (Stem Cell Technologies, Vancouver, Canada). The cells can then optionally be subjected to centrifugation to separate cells having tetrameric complexes bound thereto from all other cells. This approach depletes CD8+ T cells, B cells, DCs and other myeloid cells and results in a population of CD4+ cells from which CD4+CD25+ cells can be obtained. Subsequent manipulation to select CD4+CD25+ cells can be accomplished with positive selection (e.g., beads, magnetic particles or FACS). Alternatively, depletion of regulatory T cells can be accomplished by negative selection with antibodies to CD25, followed by affinity columns, affinity beads or magnetic particles or complement-mediated lysis.

Isolation of regulatory T cells may also embrace removal of only a subset of cultured cells. For example, in some embodiments, it may be necessary to remove CD8+ cells or B cells, or both. These are not intended to be limiting examples and those of ordinary skill in the art can apply cell selection parameters on this population as suited to a particular application.

In still other embodiments, once regulatory T cells are generated in the culture, it may be desirable to alter the culture conditions to favor their growth. For example, to the culture may be added factors that stimulate regulatory T cell proliferation such as that described in published PCT application WO 03/075953 A2.

Isolated regulatory T cells refer to a population of regulatory T cells that are substantially separated from other cells in a Cytomatrix co-culture. Substantially separated as used herein means that the regulatory T cells represent at least 50%, at least 60%, at least 70%, at least 80%, at least 85%, at least 90%, at least 95%, or at least 98% of the cells in the population.

Regulatory T cells generally represent about 30-50% of T cells in the co-culture. T cells in turn represent about 20-30% of cell in the co-culture. Accordingly, regulatory T cells represent about 5-15% of cells in the co-culture. As used herein, an enriched population of regulatory T cells is a population in which at least 20% of cells are regulatory T cells. More preferably at least 30% and even more preferably at least 40% of cells are regulatory T cells in an enriched population.

Removal of regulatory T cells can be effected in a number of ways which will be apparent to those of ordinary skill in the art. For example, regulatory T cells can be removed by FACS (i.e., removal of CD4+CD25+ and concomitant retention of all other cells). These cells can also be removed using magnetic beads or particles to isolate CD4+ cells followed by exposure to an affinity column comprising an anti-CD25 antibody. CD4+CD25+ cells would be trapped in the column. Alternatively, the affinity column step could be replaced with a complement-mediated lysis step to remove CD25+ cells.

As mentioned above, the hematopoietic progenitor cells, and progeny thereof, can be genetically altered. Genetic alteration of a hematopoietic progenitor cell includes all transient and stable changes of the cellular genetic material which are created by the addition of exogenous genetic material. Examples of genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this concept. Exogenous genetic material includes nucleic acids or oligonucleotides, either natural or synthetic, that are introduced into the hematopoietic progenitor cells. The exogenous genetic material may be a copy of that which is naturally present in the cells, or it may not be naturally found in the cells. It typically is at least a portion of a naturally occurring gene which has been placed under operable control of a promoter in a vector construct. Various techniques may be employed for introducing nucleic acids into cells and reference can be made to U.S. Pat. No. 6,548,299 B1, issued Apr. 15, 2003, for such teachings.

In still other aspects, regulatory T cells can be removed or depleted from dendritic cell based or antigen presenting cell based vaccinations since their presence would diminish any ensuing antigen specific immune response. Regulatory T cells can also be depleted from populations of antigen-specific effector T cell populations that are used clinically, for example, in combatting infectious disease in particular patient subsets.

In all of the culturing methods according to the invention, except as otherwise provided, the media used is that which is conventional for culturing cells. Examples include RPMI, DMEM, Iscove's, etc. Typically these media are supplemented with human or animal plasma or serum. As will be appreciated by one of ordinary skill in the art, preferably human serum is used to culture human progenitors and murine or bovine serum can be used to culture murine progenitors. Such plasma or serum can contain small amounts of hematopoietic growth factors. The media used according to the present invention, however, can depart from that used conventionally in the prior art.

Typically, the number of progenitor cells introduced into a Cytomatrix® culture will depend on the size and/or volume of the culture. As an example, 2.5×10⁵ CD34+ cells are routinely introduced into a culture the size of 9×1.5 mm and this leads to reproducible regulatory T cell production. (See Examples for cell numbers for larger cultures.) The number of regulatory T cells that can be produced and isolated from the Cytomatrix® cultures will similarly depend on the size and/or volume of the culture and the number of introduced progenitor cells. Typically, 10⁵ to 10⁷ (or more) regulatory T cells can be produced and isolated using the methods described herein. In a clinical setting, the cultures could be scaled up to for example 25 ml cultures that are seeded initially with 8−15×10⁶ CD34+ cells and that could generate 10⁵ to 10⁸ regulatory T cells following about two weeks of culture.

In some embodiments, the cultures do not contain stromal cell conditioned medium. “Stromal cell conditioned medium” refers to medium in which the aforementioned lymphoreticular stromal cells have been incubated. The incubation is performed for a period sufficient to allow the stromal cells to secrete factors into the medium. Although such “stromal cell conditioned medium” can be used to supplement the culture of hematopoietic progenitor cells promoting their proliferation and/or differentiation, this is not preferred according to some important embodiments of the invention.

The cell populations attained according to the methods of the invention can be used in a variety of ways.

The cultured cells may be separated into minimally two fractions: a regulatory T cell fraction and a fraction depleted of regulatory T cells. The invention provides different uses for each fraction both in vitro and in vivo. In vitro both fractions can be used to study the development of regulatory T cells, including identifying factors that control (i.e., facilitate or inhibit) such development. In vivo the fractions can be used in various clinical settings including those requiring immune inhibition or immune stimulation.

Regulatory T cells can be used in vivo to inhibit immune responses. Generally such immune responses will be abnormally elevated or alternatively their presence is inappropriate and causes more damage than benefit to a subject. These types of immune responses can take many forms such as inflammatory conditions, which as used herein include autoimmune diseases, hypersensitivity such as contact hypersensitivity or delayed type hypersensitivity, abscesses, inappropriate immune responses associated with microbial infections such as but not limited to respiratory syncitial virus, and other conditions such as but not limited to mycarditis and atherosclerosis. As used herein, “to inhibit an immune response” means to reduce or altogether eliminate the immune response. Immune response inhibition can be assessed by a reduction in the number of leukocytes in a particular location, a reduction in swelling, redness or temperature (e.g., for localized inflammatory conditions), a reduction in the level of certain cytokines in the peripheral blood of a subject (e.g., interferons including TNF and IFN-γ, IL-2, IL-4, C reactive protein, and the like), as well as general well-being of the subject.

“Inflammation” as used herein, is a localised protective response elicited by a foreign (non-self) antigen, and/or by an injury or destruction of tissue(s), which serves to destroy, dilute or sequester the foreign antigen, the injurious agent, and/or the injured tissue. Inflammation occurs when tissues are injured by viruses, bacteria, trauma, chemicals, heat, cold, or any other harmful stimuli. In such instances, the classic weapons of the immune system (T cells, B cells, macrophages) interface with cells and soluble products that are mediators of inflammatory responses (neutrophils, eosinophils, basophils, kinin and coagulation systems, and complement cascade).

The inflammatory condition may be non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis, and an abscess.

“Non-self” antigens are those antigens on substances entering a subject, or exist in a subject but are detectably different or foreign from the subject's own constituents, whereas “self” antigens are those which, in the healthy subject, are not detectably different or foreign from its own constituents. However, under certain conditions, including in certain disease states, an individual's immune system will identify its own constituents as “non-self,” and initiate an immune response against “self-antigens,” at times causing more damage or discomfort as from, for example, an invading microbe or foreign material, and often producing serious illness in a subject.

Thus, in another important embodiment, the inflammation is caused by an immune response against “self-antigen,” and the subject in need of treatment according to the invention has an autoimmune disease. “Autoimmune disease” as used herein, results when a subject's immune system attacks its own organs or tissues, producing a clinical condition associated with the destruction of that tissue, as exemplified by diseases such as autoimmune hepatitis, rheumatoid arthritis, rheumatic fever, ulcerative colitis, Crohn's disease, autoimmune inflammatory bowel disease, insulin-dependent diabetes mellitus, diabetes mellitus, juvenile diabetes, spontaneous autoimmune diabetes, gastritis, autoimmune atrophic gastritis, autoimmune hepatitis, thyroiditis, Hashimoto's thyroiditis, insulitis, oophoritis, orchitis, uveitis, phacogenic uveitis, multiple sclerosis, myasthenia gravis, primary myxoedema, thyrotoxicosis, pernicious anemia, autoimmune haemolytic anemia, Addison's disease, scleroderma, Goodpasture's syndrome, Guillain-Barre syndrome, Graves' disease, glomerulonephritis, psoriasis, pemphigus vulgaris, pemphigoid, sympathetic opthalmia, idiopathic thrombocylopenic purpura, idiopathic feucopenia, Siogren's syndrome, Wegener's granulomatosis, poly/dermatomyositis, and systemic lupus erythematosus.

Autoimmune disease may be caused by a genetic predisposition alone, by certain exogenous agents (e.g., viruses, bacteria, chemical agents, etc.), or both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of antibodies and cellular immune responses which then begin to destroy those tissues. Other autoimmune diseases develop after exposure of a subject to antigens which are antigenically similar to, that is cross-reactive with, the subject's own tissue. In rheumatic fever, for example, an antigen of the streptococcal bacterium, which causes rheumatic fever, is cross-reactive with parts of the human heart. The antibodies cannot differentiate between the bacterial antigens and the heart muscle antigens, consequently cells with either of those antigens can be destroyed.

Other autoimmune diseases, for example, insulin-dependent diabetes mellitus (involving the destruction of the insulin producing beta-cells of the islets of Langerhans), multiple sclerosis (involving the destruction of the conducting fibers of the nervous system) and rheumatoid arthritis (involving the destruction of the joint-lining tissue), are characterized as being the result of a mostly cell-mediated autoimmune response and appear to be due primarily to the action of T cells (See, Sinha et al., Science, 1990, 248:1380). Yet others, such as myesthenia gravis and systemic lupus erythematosus, are characterized as being the result of primarily a humoral autoimmune response. Nevertheless, suppression of the immune response by administering regulatory T cells either locally or systemically (as the condition requires) is beneficial to the subject since it inhibits escalation of the inflammatory response, protecting the specific site (e.g., tissue) involved, from “self-damage.”

In yet other embodiments, the inflammation is caused by an immune response against “non-self-antigens” (including antigens of necrotic self-material), and the subject in need of treatment according is a transplant recipient, has atherosclerosis, has suffered a myocardial infarction and/or an ischemic stroke, has an abscess, and/or has myocarditis. This is because after cell (or organ) transplantation, or after myocardial infarction or ischemic stroke, certain antigens from the transplanted cells (organs), or necrotic cells from the heart or the brain, can stimulate the production of immune lymphocytes and/or autoantibodies, which later participate in inflammation/rejection (in the case of a transplant), or attack cardiac or brain target cells causing inflammation and aggravating the condition (Johnson et al., Sem. Nuc. Med. 1989, 19:238:Leinonen et al., Microbiol. Path., 1990, 9:67; Montalban et al., Stroke, 1991, 22:750).

In some embodiments, the cell populations generated according to the invention can be used in transplantation settings. One major complication of transplantation is graft versus host disease (GVHD) which generally involves an attack of the host's tissues by the transplanted graft. Administration of regulatory T cells to a transplant recipient, therefore, can diminish the likelihood and/or severity of GVHD. The use of regulatory T cells can moreover reduce the dependency on immunosuppressants in allogeneic transplant settings.

In some instances, however, it is desirable to have immune responses deriving from a transplanted graft. This is the case most commonly when the subject has for example a cancer such as a leukemia and a graft versus leukemia reaction is desired. In these latter cases, it would be advisable to deplete regulatory T cells from a cell population to be transplanted.

In still other embodiments, a sufficient number of regulatory T cells are administered together with a graft in order to provide a beneficial graft versus disease response without inducing detrimental GVHD. Thus, the invention allows for the administration of a pre-determined ratio of effector:regulatory T cells according to the condition of the subject. The ratio of regulatory T cells to effector T cells may vary and can include but is not limited to 1:2, 1:5, 1:10, 1:100 or 1:1000.

As used herein, a subject is a human, non-human primate, cow, horse, pig, sheep, goat, dog, cat or rodent. Cultures employing human hematopoietic progenitor cells and human subjects are particularly important embodiments. Cultures aimed at generation of murine regulatory T cells are also important as such cells can be used to study the development and factor responsiveness of such cells, as well as in screening assays for agents that modulate their development, expansion and exhaustion.

In some aspects of the invention, the cultures are performed in the presence of antigens and/or antigen presenting cells. These cultures in some embodiments preferably comprise effector T cells. Culture with antigen and/or antigen presenting cells may be concurrent to culture of progenitor and stromal cells, or it may be subsequent to such culture. In the case of antigen presenting cells, such cells may be in an immature form and may mature during the co-culture.

Culture in the presence of APCs may also include a co-stimulatory agent. Co-stimulatory agents include lymphocyte function associated antigen-3 (LFA-3), CD2, CD40, CD80/B7-1, CD86/B7-2, OX-2, CD70, and CD82. Co-stimulatory agents may also be used in lieu of APCs, provided that MHC class II molecules and anti-CD3 antibodies are co-administered with the co-stimulatory agent(s).

The regulatory T cells may develop as a result of antigen exposure or alternatively they may develop concurrently with antigen-specific effector T cells. Antigen-specific effector T cells have been used clinically in subjects at risk of developing or subjects having particular infectious disease. These subjects include but are not limited to subjects undergoing hematopoietic reconstitution (e.g., those undergoing bone marrow transplantation). As an example, these latter subjects are particularly prone to CMV infections. Autologous progenitors, including T cell committed cells, are harvested from the peripheral blood of subjects and exposed to antigen in vitro (e.g., in the Cytomatrix® cultures of the invention but not so limited), in order to generate a population of antigen-specific effector T cells. The nature of the antigen is non-limiting and examples are provided below. In some embodiments, these antigens derive from CMV, EBV, Hepatitis virus and HIV. Populations of antigen-specific effector T cells can be depleted of regulatory T cells, for example according to any of the methods described herein. Depletion of regulatory T cells from these populations will increase the likelihood that a therapeutically beneficial immune response will be mounted in the subject following administration.

An antigen, as used herein, falls into four classes: 1) antigens that are characteristic of a pathogen; 2) antigens that are characteristic of an autoimmune disease; 3) antigens that are characteristic of an allergen; and 4) antigens that are characteristic of a tumor. Antigens in general include polysaccharides, glycolipids, glycoproteins, peptides, proteins, carbohydrates and lipids from cell surfaces, cytoplasm, nuclei, mitochondria and the like.

Antigens that are characteristic of pathogens include antigens derived from viruses, bacteria, parasites or fungi. Examples of important pathogens include vibrio choleras, enterotoxigenic Escherichia coli, rotavirus, Clostridium difficile, Shigella species, Salmonella typhi, parainfluenza virus, influenza virus, Streptococcus pneumonias, Borella burgdorferi, HIV, Streptococcus mutans, Plasmodium falciparum, Staphylococcus aureus, rabies virus and Epstein-Barr virus.

Viruses in general include but are not limited to those in the following families: picornaviridae; caliciviridae; togaviridae; flaviviridae; coronaviridae; rhabdoviridae; filoviridae; paramyxoviridae; orthomyxoviridae; bunyaviridae; arenaviridae; reoviridae; retroviridae; hepadnaviridae; parvoviridae; papovaviridae; adenoviridae; herpesviridae; and poxyviridae; and viruses including, but not limited to, cytomegalovirus; Hepatitis A,B,C, D, E; Herpes simplex virus types 1& 2; Influenzae virus; Mumps virus; Parainfluenza 1, 2 and 3; Epstein Barr virus; Respiratory syncytial virus; Rubella virus; Rubeola virus; Varicella-zoster virus; Vibrio Cholerae; Human immunodeficiency viruses (HIVs) and HIV peptides, including HIV-1 gag, HIV-1 env, HIV-2 gag, HIV-2 env, Nef, RT, Rev, gp120, gp41, p15, p17, p24, p7-p6, Pol, Tat, Vpr, Vif, Vpu; Hantavirus; Ebola virus; Lymphocytic ChorioMeningitis virus; Dengue virus; Rotavirus; Human T-lymphotropic (HTLV-I); HTLV-II; Human herpesvirus-6 (HHV-6); HHV-8; Guanarito virus; Bartonella henselae; Sin nombre virus; and Sabia virus. Exemplary cytomegalovirus epitopes include GP 33-43, NP396-404, and GP276-286. An exemplary influenza epitope includes the HA peptide.

Bacteria in general include but are not limited to: P. aeruginosa; Bacillus anthracis; E. coli, Enterocytozoon bieneusi; Klebsiella sp.; Klebsiella pneumoniae; Serratia sp.; Pseudomonas sp.; P. cepacia; Acinetobacter sp.; S. epidermis; E. faecalis; S. pneumoniae; S. aureus; Haemophilus sp.; Haemophilus Influenza; Neisseria Sp.; Neisseria gonorheae; Neisseria meningitis; Helicobacter pylori; Bacteroides sp.; Citrobacter sp.; Branhamella sp.; Salmonella sp.; Salmonella typhi; Shigella sp.; S. pyogenes; Proteus sp.; Clostridium sp.; Erysipelothrix sp.; Lesteria sp.; Pasteurella multocida; Streptobacillus sp.; Spirillum sp.; Fusospirocheta sp.; Actinomycetes; Mycoplasma sp.; Chlamydiae sp.; Chlamydia trachomatis; Campylobacterjejuni; Cyclospora cayatanensis; Rickettsia sp.; Spirochaeta, including Treponema pallidum and Borrelia sp.; Legionella sp.; Legionella pneumophila; Mycobacteria sp.; Mycobacterium tuberculosis; Ureaplasma sp.; Streptomyces sp.; Trichomonas sp.; and P. mirabilis, as well as toxins, that include, but are not limited to, Anthrax toxin (EF); Adenylate cyclase toxin; Cholera enterotoxin; E. coli LT toxin; Escherichia coli 0157:H7; Shiga toxin; Botulinum Neurotoxin Type A heavy and light chains; Botulinum Neurotoxin Type B heavy and light chains; Tetanus toxin; Tetanus toxin C fragment; Diphtheria toxin; Pertussis toxin; Parvovirus B19; Staphylococcus enterotoxins; Toxic shock syndrome toxin (TSST-1); Erythrogenic toxin; and Vibrio cholerae 0139.

Parasites include but are not limited to: Ehrlichia chafeensis; Babesia; Encephalitozoon cuniculi; Encephalitozoon hellem; Schistosoms; Toxoplasma gondii; Plasmodium falciparum, P. vivax, P. ovale, P. malaria; Toxoplasma gondii; Leishmania mexicana, L. tropica, L. major, L. aethiopica, L. donovani, Trypanosoma cruzi, T. brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T. saginata, Trichomonas vaginatis, T. hominis, T. tenax; Giardia lamblia; Cryptosporidum parvum; Pneumocytis carinii, Babesia bovis, B. divergens, B. microti, Isospore belli, L hominis; Dientamoeba fragiles; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirius pubis; and Dermatobia hominis.

Fungi in general include but are not limited to: Cryptococcus neoformans; Blastomyces dermatitidis; Aiellomyces dermatitidis; Histoplasfria capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; and Dermatophyres species.

Antigens that are characteristic of autoimmune disease typically will be derived from the cell surface, cytoplasm, nucleus, mitochondria and the like of mammalian tissues. Examples include antigens characteristic of uveitis (e.g. S antigen), diabetes mellitus, multiple sclerosis, systemic lupus erythematosus, Hashimoto's thyroiditis, myasthenia gravis, primary myxoedema, thyrotoxicosis, rheumatoid arthritis, pernicious anemia, Addison's disease, scleroderma, autoimmune atrophic gastritis, premature menopause (few cases), male infertility (few cases), juvenile diabetes, Goodpasture's syndrome, pemphigus vulgaris, pemphigoid, sympathetic opthalmia, phacogenic uveitis, autoimmune haemolytic anemia, idiopathic thrombocylopenic purpura, idiopathic feucopenia, primary biliary cirrhosis (few cases), ulcerative colitis, Siogren's syndrome, Wegener's granulomatosis, poly/dermatomyositis, and discold lupus erythromatosus.

The invention can also be used in the treatment or prevention of allergies, as well as in the treatment of asthma. Allergic conditions or diseases in humans include but are not limited to eczema, allergic rhinitis or coryza, hay fever, conjunctivitis, bronchial or allergic asthma, urticaria (hives) and food allergies; atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many allergens, however, are protein or polypeptide in nature, as proteins and polypeptides are generally more antigenic than carbohydrates or fats. Allergens may also be low molecular weight allergenic haptens that induce allergy after covalently combining with a protein carrier (Remington's Pharmaceutical Sciences). Common allergens include antigens derived from pollens, dust, molds, spores, dander, insects and foods. Specific examples include the urushiols (pentadecylcatechol or heptadecyicatechol) of Toxicodendron species such as poison ivy, poison oak and poison sumac, and the sesquiterpenoid lactones of ragweed and related plants.

Allergens also include but are not limited to Environmental Aeroallergens; plant pollens such as Ragweed/hayfever; Weed pollen allergens; Grass pollen allergens; Johnson grass; Tree pollen allergens; Ryegrass; House dust mite allergens; Storage mite allergens; Japanese cedar pollen/hay fever Mold spore allergens; Animal allergens (cat, dog, guinea pig, hamster, gerbil, rat, mouse); Food Allergens (e.g., Crustaceans; nuts, such as peanuts; citrus fruits); Insect Allergens (Other than mites listed above); Venoms: (Hymenoptera, yellow jacket, honey bee, wasp, hornet, fire ant); Other environmental insect allergens from cockroaches, fleas, mosquitoes, etc.; Bacteria such as streptococcal antigens; Parasites such as Ascaris antigen; Viral Antigens; Fungal spores; Drug Allergens; Antibiotics; penicillins and related compounds; other antibiotics; Whole Proteins such as hormones (insulin), enzymes (Streptokinase); all drugs and their metabolites capable of acting as incomplete antigens or haptens; Industrial Chemicals and metabolites capable of acting as haptens and stimulating the immune system (Examples are the acid anhydrides (such as trimellitic anhydride) and the isocyanates (such as toluene diisocyanate)); Occupational Allergens such as flour (i.e. Baker's asthma), castor bean, coffee bean, and industrial chemicals described above; flea allergens; and human proteins in non-human animals.

Examples of specific natural, animal and plant allergens include but are not limited to proteins specific to the following genuses: Canine (Canisfamiliaris); Dermatophagoides (e.g. Dermatophagoidesfarinae); Felis (Felis domesticus); Ambrosia (Ambrosia artemiisfolia; Lolium (e.g. Lolium perenne or Lolium multiflorum); Cryptomeria (Cryptomeriajaponica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

The regulatory T cells can also be used as an adjunct treatment in subjects that demonstrate (or are likely to demonstrate) allergic reactions to particular therapeutic agents or regimens. For example, subjects having allergies to particular drugs (e.g., penicillin) may be administered regulatory T cells once or repeatedly in order to control the allergic reaction and thus facilitate the administration of the drug. Similarly, subjects undergoing transfusions or pheresis regularly are also candidates for administration of regulatory T cells, particularly if they are susceptible to experiencing an adverse reaction to the transfusion or pheresis procedure. These subjects may be administered the regulatory T cells in anticipation of an allergic reaction causing event such as a transfusion. The regulatory T cells may be administered one week or 6, 5, 4, 3, 2, or 1 day or less than 12, less than 4, less than 2 hours prior to transfusion, for example.

Antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and tumor mucins and glycolipids. Tumors include, but are not limited to, those from the following sites of cancer and types of cancer: biliary tract cancer; brain cancer, including glioblastomas and medulloblastomas; breast cancer; cervical cancer; choriocarcinoma; colon cancer; endometrial cancer; esophageal cancer; gastric cancer; hematological neoplasms, including acute lymphocytic and myelogeneous leukemia; multiple myeloma; AIDS associates leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basal cell cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. Viral proteins associated with tumors would be those from the classes of viruses noted above. Antigens characteristic of tumors may be proteins not usually expressed by a tumor precursor cell, or may be a protein which is normally expressed in a tumor precursor cell, but having a mutation characteristic of a tumor. An antigen characteristic of a tumor may be a mutant variant of the normal protein-having an altered activity or subcellular distribution. Mutations of genes giving rise to tumor antigens, in addition to those specified above, may be in the coding region, 5′ or 3′ noncoding regions, or introns of a gene, and may be the result of point mutations frameshifts, deletions, additions, duplications, chromosomal rearrangements and the like. One of ordinary skill in the art is familiar with the broad variety of alterations to normal gene structure and expression which gives rise to tumor antigens.

Specific examples of tumor antigens include: proteins such as Ig-idiotype of B cell lymphoma, mutant cyclin-dependent kinase 4 of melanoma, Pmel-17 (gp 100) of melanoma, MART-1 (Melan-A) of melanoma, p15 protein of melanoma, tyrosinase of melanoma, MAGE 1, 2 and 3 of melanoma, thyroid medullary, small cell lung cancer, colon and/or bronchial squamous cell cancer, BAGE of bladder, melanoma, breast, and squamous-cell carcinoma, gp75 of melanoma, oncofetal antigen of melanoma; carbohydrate/lipids such as muci mucin of breast, pancreas, and ovarian cancer, GM2 and GD2 gangliosides of melanoma; oncogenes such as mutant p53 of carcinoma, mutant ras of colon cancer and HER21neu proto-onco-gene of breast carcinoma; viral products such as human papilloma virus proteins of squamous cell cancers of cervix and esophagus; and antigens (shown in parenthesis) from the following tumors: acute lymphoblastic leukemia (etv6; am11; cyclophilin b), glioma (E-cadherin; .alpha.-catenin; .beta.-catenin; .gamma.-catenin; p120ctn), bladder cancer (p21ras), billiary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)-CO17-1A/GA733; APC), choriocarcinoma (CEA), epithelial cell-cancer (cyclophilin b), gastric cancer (HER2/neu; c-erbB-2; ga733 glycoprotein), hepatocellular cancer (.alpha.-fetoprotein), hodgkins lymphoma (lEBNA-1), lung cancer (CEA; MAGE-3; NY-ESO-1), lymphoid cell-derived leukemia (cyclophilin b), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), testicular cancer (NY-ESO-1), T cell leukemia (HTLV-1 epitopes), and melanoma (Melan-A/MART-1; cdc27; MAGE-3; p21ras; gp100.sup.Pmel 117). It is also contemplated that proteinaceous tumor antigens may be presented by HLA molecules as specific peptides derived from the whole protein. Metabolic processing of proteins to yield antigenic peptides is well known in the art; for example see U.S. Pat. No. 5,342,774 (Boon et al.). and the ones on the lists previously.

Antigens may also include: C reactive protein; Coxsackie B1, B2, B3, B4, EI5, B6 proteins; Myelin basic protein; pancreatic beta-cell antigens; arthritis associated antigens (cartilage, aggrecan, type II collagen); AP-1; NF-kappaB; desmoglein (Dsg 1 or 3); and alzheimer's associated antigens (prions, amyloid-beta protein), and/or any synthetic agent that binds to the T-cell receptor.

Further exemplary cancer, viral, and beta islet autoantigens are described below in Tables 1, 2 and 3 respectively.

TABLE 1
Exemplary Cancer Antigens

| | | | | |
|---|---|---|---|---|
| | | | | SEQ |
| | | | | ID |
| Protein | MHC | Peptide | Position | NO: |

| | | | | |
|---|---|---|---|---|
| MAGE-A1 | HLA-A1 | EADPTGHSY | 161-169 | 1 |
| | HLA-Cw16 | SAYGEPRKL | 230-238 | 2 |
| MAGE-A3 | HLA-A1 | EVDPIGHLY | 168-176 | 3 |
| | HLA-A2 | FLWGPRALV | 271-279 | 4 |
| | HLA-B44 | MEVDPIGHLY | 167-176 | 5 |
| MAGE-A6 | HLA-Cw16 | KISGGPRISYPL | 292-303 | 6 |
| MAGE | | ALSRKVAEL | | 7 |
| melanoma AG | | | | |
| BAGE | HLA-Cw16 | AARAVFLAL | 2-10 | 8 |
| GAGE-1,2 | HLA-Cw16 | YRPRPRRY | 9-16 | 9 |
| RAGE | HLA-B7 | SPSSNRIRNT | 11-20 | 10 |
| GnT-V | HLA-A2 | VLPDVFIRC | 2-10/11 | 11 |
| MUM-1 | HLA-B44 | EEKLIVVLF | exon | 12 |
| | | | 2/intron | |
| | | EEKLSVVLF | | 13 |
| | | (wild type) | | |
| CDK4 | HLA-A2 | ACDPHSGHFV | 23-32 | 14 |
| | | ARDPHSGHFV | | 15 |
| | | (wild type) | | |
| β-catenin | HLA-A24 | SYLDSGIHF | 29-37 | 16 |
| | | SYLDSGIHS | | 17 |
| | | (wild type) | | |
| Tyrosinase | HLA-A2 | MLLAVLYCL | 1-9 | 18 |
| | HLA-A2 | YMNGTMSQV | 369-377 | 19 |
| | HLA-A2 | YMDGTMSQV | 369-377 | 20 |
| | HLA-A24 | AFLPWHRLF | 206-214 | 21 |
| | HLA-B44 | SEIWRDIDF | 192-200 | 22 |
| | HLA-B44 | YEIWRDIDF | 192-200 | 23 |
| | HLA-DR4 | QNILLSNAPLGPQFP | 56-70 | 24 |
| | HLA-DR4 | DYSYLQDSDPDSFQD | 448-462 | 25 |
| | HLA-A2 | ILTVILGVL | 32-40 | 26 |
| gp100^{Pmel117} | HLA-A2 | KTWGQYWQV | 154-162 | 27 |
| | HLA-A2 | ITDQVPFSV | 209-217 | 28 |
| | HLA-A2 | YLEPGPVTA | 280-288 | 29 |
| | HLA-A2 | LLDGTATLRL | 457-466 | 30 |
| | HLA-A2 | VLYRYGSFSV | 476-485 | 31 |
| PRAME | HLA-A24 | LYVDSLFFL | 301-309 | 32 |
| NY-ESO-1 | HLA-A2 | SLLMWITQCFL | 157-167 | 33 |
| | HLA-A2 | SLLMWITQC | 157-165 | 34 |
| | HLA-A2 | QLSLLMWIT | 155-163 | 35 |
| c-erb2 | | HLYQGCQVVPLTSIIS | | 36 |
| | | AV | | |
| p53 | 264-272 | LLGRNSFEV | | 37 |

TABLE 2
Exemplary Viral Antigens

| | | | | SEQ |
|---|---|---|---|---|
| | | | | ID |
| Protein | MHC | Peptide | Position | NO: |
| Rubella E1 | | WVTPVIGSQARKCGL | 276-290 | 38 |
| | | RVIDPAAQ | 412-419 | 39 |
| Measles F | | HQALVIKLMPNITLL | | 40 |
| Papilloma | | RLCVQSTHV | | 41 |
| | | YVRDGNPYA | E6 60-68 | 42 |
| | | GYNKPLCDLL | E6 98-107 | 43 |
| Influenza | | KGILGFVFTLTV | 57-68 | 44 |
| matrix | | | | |
| Influenza | | EKYVKQNTLKLAT | 307-319 | 45 |
| HA | | | | |
| Hepatitis B | | WLSLLVPFV | | 46 |
| SAg | | | | |
| | | FLGGTTVCL | | 47 |
| Hepatitis C | | YLVAYQATV | | 48 |
| NS | | | | |
| NS3 | | GLRDLAVAV | | 49 |
| | | GYKVLVLNPSVAAT | 1248-1261 | 50 |
| | | KLVALGINAV | 1406-1415 | 51 |
| Tetanus | | QYIKANSKFIGIYQL | 830-843 | 52 |

TABLE 3
Exemplary Beta Islet Cell Autoantigens:

| | | | SEQ |
|---|---|---|---|
| | | | ID |
| Protein | Peptide | Position | NO: |
| glutamic acid | TYELAPVFVLLEYVT | 206-220 | 53 |
| decarboxylase 65 | | | |
| | LKKMRFIIGWPGGSG | 221-235 | 54 |
| | KKGAAAIGIGTDSVI | 286-300 | 55 |
| | PLQCSALLVREEGLM | 401-415 | 56 |
| | WLMWRAKGTTGFEAH | 456-470 | 57 |
| tyrosine | VIVMLTPLVEDGVKQC | 805-820 | 58 |
| phosphatase | | | |
| IA-2 | | | |

The cell populations, as described above, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result. In some cases this is a local (site-specific) reduction of inflammation.

A variety of administration routes are available. The methods of the invention, generally speaking may be practiced using any mode of administration that is medically acceptable, meaning any mode that produces effective levels of the particular cell populations without causing clinically unacceptable adverse effects. Such modes of administration include oral, rectal, topical, nasal, interdermal, or parenteral routes. The term “parenteral” includes subcutaneous, intravenous, intramuscular, or infusion. Local administrations encompass intra-joint administration (e.g., via injection), intramuscular administration, intraspinal administration, administration via vessels that drain into the pancreas such as the anterior and posterior pancreatico duodenal arteries, and the like.

Regulatory T cells may be administered to a subject once or repeatedly. As used herein, repeated administration includes twice a week, weekly, biweekly, monthly, bimonthly, etc. Repeated administrations may be provided in anticipation of an abnormal or inappropriate immune response such as may occur when for example immunosuppressant medication is at reduced levels in the body of a subject.

In still another aspect, the invention provides screening methods and systems for the identification of agents that influence regulatory T cell development and/or generation. The screening methods generally require a control and a test culture system. The control culture system is a positive control. The test culture system may have more, less or equal numbers of regulatory T cells depending upon whether the candidate agent is an inhibitor or a stimulator of regulatory T cell production, or whether it has no effect on regulatory T cell production at all. Candidate agents may be derived from natural sources, including but not limited to cell culture supernatants, bodily fluids (particularly from subjects having chronic abnormal immune responses), and the like. Alternatively, candidate agents may be derived from synthetic sources, such as synthetic (preferably small molecule) libraries. The screening methods are not intended to be so limited, however.

The invention will be more fully understood by reference to the following examples. These examples, however, are merely intended to illustrate the embodiments of the invention and are not to be construed to limit the scope of the invention.

### EXAMPLES

### Primary Stromal Cultures:

Human thymus and skin were obtained from donor banks (CHTN, Philadelphia, Pa. or NDRI, Philadelphia, Pa.) and mechanically dissociated into 2 mm³ fragments, 3-4 fragments were placed directly on the surface of Cytomatrix® units (9×1.5 mm) (Cytomatrix, Woburn, Mass.). Matrices were cultured in 48-well tissue culture plates in IMDM (JRH Biosciences, Lenexa, Kans.) supplemented with 10% human serum (Sigma, St. Louis, Mo.), glutamine (1 lmM), penicillin (10 IU/ml), streptomycin (10 mg/ml) (Life Technologies, Rockville, Md.). Similar experiments were performed in 14×1.5 mm Cytomatrix® units cultured in 24 well plates. Matrix units were completely submersed in media and medium was changed twice weekly. All cultures were incubated at 37° C. in 5% C0₂ for 2 weeks. Adherent cells were not irradiated or treated with mitomycin C.

Progenitor cells (CD34+) were then added to the thymus or skin cultures in the Cytomatrix® units in the absence or presence of exogenous IL-15 (20 ng/ml) and IL-7 (20 ng/ml). 250,000 progenitors were added to the 9×1.5 mm Cytomatrix® units and between 500,000 and 2×10⁶ progenitor cells in the 14×1.5 mm Cytomatrix® units. Non-adherent cells were analyzed after 14 days for phenotypic expression of lymphoid markers by staining with fluorescent antibodies and analyzing a FACSCalibur flow cytometer (Becton Dickinson, San Jose, Calif.) and FloJo software.

### Isolation of Hematopoietic Progenitors:

For these experiments, CD34 cells were isolated with a research based device (Miltenyi MiniMacs, Miltenyi, Auburn, Calif.) or a clinical grade isolation system (Baxter Isolex, distributed by Miltenyi, Auburn, Calif.). Both freshly isolated CD34+ cells as well as cryopreserved CD34+ cells have been used with no measurable difference in performance. For cryopreservation, CD34+ isolated cells were frozen in aliquots in 90% FBS (Sigma, St. Louis, Mo.), and 10% DMSO (Sigma, St. Louis, Mo.) and stored in liquid nitrogen.

i) Isolation of Cells Using MiniMacs:

CD34⁺cells from UCB, bone marrow or peripheral blood mobilized stem cells (AllCells, Berkely, Calif. or Cambrex, Rockland, Md.) were isolated by immuno-magnetic column separation following the manufacturers protocol (Miltenyi Biotec, Auburn, Calif.). CD34 purity was evaluated using FACS, progenitor cells used in subsequent assays were qualified as having <1% contaminating T cells.

ii) Isolation of Cells Using Isolex:

Peripheral blood mononuclear cells were obtained via leukapheresis from normal human volunteers (AllCells, Berkely, Calif.). CD34+ cells were purified using an Isolex 300 SA device according to the manufacturers instructions. CD34 purity was evaluated using FACS, progenitor cells used in subsequent assays were qualified as having <1% contaminating T cells. If cells were determined to have >1% contaminating T cells, a subsequent T cell depletion was performed using immuno-magnetic column separation to remove CD3+ cells following the manufacturers protocol (Miltenyi Biotec, Auburn, Calif.). Cells were used freshly isolated in subsequent experiments or frozen in aliquots in 90% FBS (Sigma, St. Louis, Mo.), and 10% DMSO (Sigma, St. Louis, Mo.) and stored in liquid nitrogen until later use.

### Assessment of Immunophenotype of Cells Derived From the Co-Cultures:

Adherent cells were harvested by washing followed by centrifugation. The Cytomatrix® cultures were manually flushed to release non-adherent cells into the media and then the Cytomatrix® units were also centrifuged at 1500 rpm for 10 minutes. Harvested cells were counted and assessed for viability by trypan blue exclusion. After counting, cells were stained in a final volume of 100 uL with 2% mouse serum (Dako, Carpentiera, Calif.) and the following fluorochrome-conjugated antibodies: TCR.alpha . . . beta., TCR.gamma . . . delta., CD2, CD3, CD4, CD8, CD14, CD25, CD45RO, CD33 and CD34(Becton Dickinson, San Jose, Calif.). Conjugated isotype control antibodies for all four fluorochromes (FITC, PE, Peridinin chlorophyll protein (PerCP), and Allophycocyanin (APC) were used for each culture. Stained samples were washed three times with PBS, fixed with 1% paraformaldehyde, and analyzed with a FACScalibur flow cytometer (Becton Dickinson). Appropriate controls included matched isotype antibodies to establish positive and negative quadrants, as well as appropriate single color stains to establish compensation. For each sample, at least 10,000 list mode events were collected.

### Isolation of CD4+CD25+ Cells:

CD4+CD25+ may be isolated from the remaining cultured cells. Two strategies for accomplishing this are described below. These strategies are not intended to be limiting.

i) Isolation of CD4+CD25+ Regulatory T Cells Produced in the Cytomatrix® System Using Immunomagnetic Beads:

Mononuclear cells isolated from the Cytomatrix® cultures as described above were subsequently fractionated using immunomagnetic beads specific for CD4 and CD25 following the manufacturers protocol (Miltenyi Biotec, Auburn, Calif.). CD4CD25 purity was evaluated using FACS, which demonstrated a purity of >80%.

ii) Isolation of CD4+CD25+ Regulatory T Cells Produced in the Cytomatrix® System Using FACS:

Mononuclear cells isolated from the Cytomatrix® cultures as described above were stained with fluorescent labeled antibodies as described above and CD4+CD25+ cells were sorted using a FACSVantage Instrument (Becton Dickinson, San Jose, Calif.) according to standard protocols. Cells isolated using this approach demonstrated a greater than 95% purity for CD4+CD25+ cells.

### Identification of CD4+CD25+ Cells:

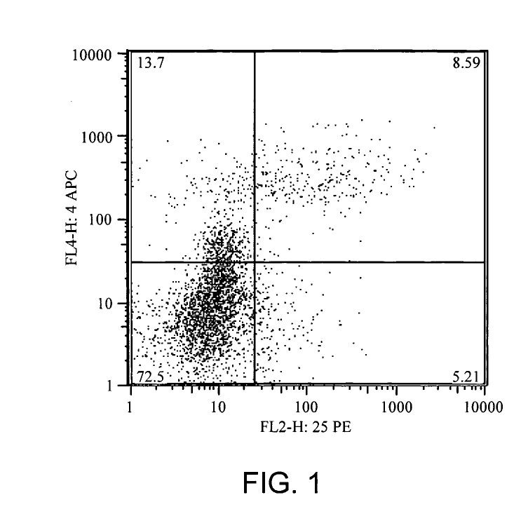

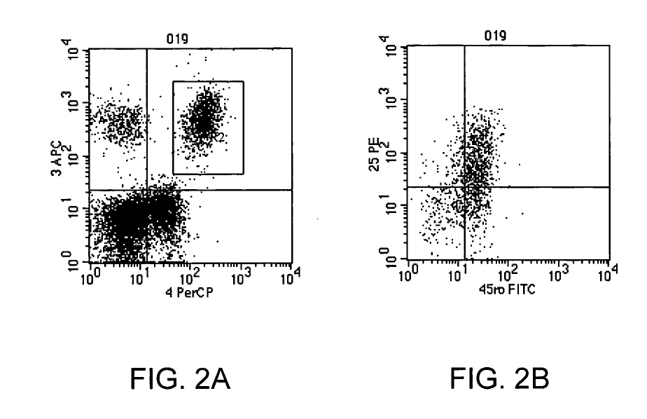

Harvest and analysis of cells from the Cytomatrix® co-cultures demonstrated that the cultures contain a variable percentage (30%-70%) of CD4+CD25+ cells (FIG. 1). The cultures contain other T cell and myeloid progeny, consistent with previous descriptions. Further analysis of the CD4+CD25+ cells demonstrated that the large majority also co-expressed the CD45RO antigen which has also been described on regulatory T cells (FIG. 2). Although CD45RO is generally expressed on memory T cells, recent thymic emigrants that are CD45RO+ have been shown to be antigen naive and in fact regulatory T cells. Cells of this phenotype have been described in both human and murine systems, and have been demonstrated in murine transplant models to confer aspects of immune regulation and participate in suppression of various autoimmune like phenomena, possibly due to the production of soluble factors that suppress effector T cells.

### EQUIVALENTS

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

We claim:

1. A method for in vitro production of regulatory T cells, comprising:
introducing an amount of hematopoietic progenitor cells and an amount of lymphoreticular stromal cells capable of mitosis into an open cell porous, solid matrix having interconnected pores of a pore size sufficient to permit the hematopoietic progenitor cells and the lymphoreticular stromal cells to grow in the matrix,
co-culturing the hematopoietic progenitor cells and the lymphoreticular stromal cells, and
isolating regulatory T cells from the cultured cells,
wherein the lymphoreticular stromal cells are derived from at least one lymphoid soft tissue selected from the group consisting of thymus, spleen, liver, lymph node, skin, tonsil, Peyer's patches and combinations thereof, and comprise one of more of fibroblasts, keratinocytes, epithelial cells, dendritic cells (DCs), and antigen presenting cells; and the amount of the lymphoreticular stromal cells is sufficient to support the growth and differentiation of the hematopoietic progenitor cells.

2. The method of claim 1, wherein the hematopoietic progenitor cells and the lymphoreticular stromal cells are co-cultured in the presence of IL-7 and IL-15.

3. The method of claim 1, wherein the hematopoietic progenitor cells and the lymphoreticular stromal cells are of human origin.

4. The method of claim 1, wherein the hematopoietic progenitor cells and the lymphoreticular stromal cells are of murine origin.

5. The method of claim 1, wherein the regulatory T cells are isolated based on CD4+CD25+ phenotype.

6. The method of claim 1, wherein the regulatory T cells are isolated using fluorescent activated cell sorting, affinity column separation, affinity magnetic beads, affinity magnetic particles, complement-mediated lysis, panning, or tetrameric complex based separation.

7. The method of claim 1, wherein the hematopoietic progenitor cells are selected from the group consisting of pluripotent stem cells, multipotent progenitor cells and progenitor cells committed to specific hematopoietic lineages.

8. The method of claim 1, wherein the hematopoietic progenitor cells are derived from tissue selected from the group consisting of bone marrow, peripheral blood, mobilized peripheral blood, umbilical cord blood, placental blood, lymphoid soft tissue, fetal liver, embryonic cells and aortal-gonadal-mesonephros derived cells.

9. The method of claim 8, wherein the hematopoietic progenitor cells are derived from tissue selected from the group consisting of bone marrow, mobilized peripheral blood and umbilical cord blood.

10. The method of claim 1, wherein the lymphoreticular stromal cells are seeded prior to inoculating the hematopoietic progenitor cells.

11. The method of claim 1, wherein the porous solid matrix is an open cell porous matrix having a percent open space of at least 75%.

12. The method of claim 1, wherein the porous solid matrix is an open cell porous matrix having at least 80 pores per square inch (ppi).

13. The method of claim 11, wherein the porous solid matrix has pores defined by interconnecting ligaments having a diameter at midpoint, on average, of less than 150 μm.

14. The method of claim 1, wherein the porous, solid matrix having seeded hematopoietic progenitor cells and their progeny, and lymphoreticular stromal cells, is impregnated with a gelatinous agent that occupies pores of the matrix.

15. The method of claim 1, wherein the lymphoid soft tissue is selected from the group consisting of thymus and skin.

16. The method of claim 15, wherein the progenitor cells committed to specific hematopoietic lineages are committed to a T cell lineage.

17. The method of claim 1, wherein the hematopoietic progenitor cells are CD34+ cells.

18. The method of claim 1, wherein the progenitor cells are CD34+ cells, the lymphoreticular stromal cells are derived from skin, and the co-culture comprises IL-7 and IL-15.

19. The method of claim 3, wherein the hematopoietic progenitor cells and the lymphoreticular stromal cells are autologous to a subject to be treated with the isolated regulatory T cells.

20. The method of claim 3, wherein the hematopoietic progenitor cells are allogeneic and the lymphoreticular stromal cells are autologous to a subject to be treated with the isolated regulatory T cells.

21. The method of claim 11, wherein the porous solid matrix is a metal-coated reticulated open cell foam of carbon containing material.

22. The method of claim 21, wherein the metal is selected from the group consisting of tantalum, titanium, platinum, niobium, hafnium, tungsten, and combinations thereof, and wherein said metal is coated with a biological agent selected from the group consisting of collagens, fibronectins, laminins, integrins, glycosaminoglycans, vitrogen, antibodies and fragments thereof, and combinations thereof.

23. The method of claim 22, wherein the metal is tantalum.

24. A method for producing a hematopoietic cell population depleted of regulatory T cells, comprising:
introducing an amount of hematopoietic progenitor cells and an amount of lymphoreticular stromal cells capable of mitosis into an open cell porous, solid matrix having interconnected pores of a pore size sufficient to permit the hematopoietic progenitor cells and the lymphoreticular stromal cells to grow in the matrix,
co-culturing the hematopoietic progenitor cells and the lymphoreticular stromal cells, and
removing regulatory T cells from the cultured cells to produce a hematopoietic cell population depleted of regulatory T cells,
wherein the lymphoreticular stromal cells are derived from at least one lymphoid soft tissue selected from the group consisting of thymus, spleen, liver, lymph node, skin, tonsil, Peyer's patches and combinations thereof, and comprises one or more of fibroblasts, keratinocytes, epithelial cells, dendritic cells (DCs), and antigen presenting cells; and the amount of the lymphoreticular stromal cells is sufficient to support the growth and differentiation of the hematopoietic progenitor cells.

25-50. (canceled)

51. A method for inhibiting an immune response, comprising
administering to a subject in need thereof isolated regulatory T cells produced according to claim 1, in an amount effective to inhibit an immune response.

52-73. (canceled)

74. A method for increasing immune reactivity of a transplanted cell population, comprising
administering to a subject in need thereof a cell population depleted of regulatory T cells.

75-79. (canceled)

80. An isolated population of regulatory T cells produced by the method of claim 1.
